(19) United States
(12) Patent Application Publication
(10) Pub. No.: US 2023/0079471 A1
(43) Pub. Date: Mar. 16, 2023

(54) Implantable Medical Device Configured for Detecting a Presence of an MRI Device

(71) Applicant: BIOTRONIK SE & Co. KG, Berlin (DE)

(72) Inventor: Uwe KAMENZ, Berlin (DE)

(73) Assignee: BIOTRONIK SE & Co. KG, Berlin (DE)

(21) Appl. No.: 17/799,087

(22) PCT Filed: Jan. 28, 2021
(86) PCT No.: PCT/EP2021/051974

§ 371 Date: Aug. 11, 2022

(30) Foreign Application Priority Data
Feb. 21, 2020 (EP) 20158713.6

(51) Int. Cl. A61N 1/37; A61N 1/39

(52) U.S. Cl. CPC A61N 1/3718; A61N 1/3925

(57) ABSTRACT

An implantable medical device comprises a sensing device for sensing a magnetic field, and a processing device configured to detect a presence of an MRI device based on measurement values obtained from the sensing device. The processing device is configured to conclude that an MRI device is present if a multiplicity of measurement values indicates an increase of a strength of the magnetic field. Further, the sensing device is configured to conduct measurements at a specified frequency in between 1 Hz and 50 Hz, in particular at 4 Hz, and to provide measurement values at a predefined sampling rate.

### CROSS-REFERENCE TO RELATED APPLICATIONS

This application is the United States National Phase under 35 U.S.C. § 371 of PCT International Patent Application No. PCT/EP2021/051974, filed on Jan. 28, 2021, which claims the benefit of European Patent Application No. 20158713.6, filed on Feb. 21, 2020, the disclosures of which are hereby incorporated by reference herein in their entireties.

### TECHNICAL FIELD

The present invention relates to an implantable medical device according to the preamble of claim 1 and to a method for operating an implantable medical device.

### BACKGROUND

An implantable medical device shall be configured for implantation into a patient. An implantable medical device in this context may, for example, be a pacemaker device for providing for a pacing action in a patient's heart, or a defibrillator device, such as an implantable cardioverter-defibrillator (ICD), for providing for a defibrillation, or a sensor device having a sensing function for monitoring, for example, a cardiac activity of a patient. In addition, the implantable medical device may be a recording device, such as a loop recorder, or a transmitter device for transmitting signals to a device external to a patient, or a data carrier, such as a patient identification device.

An implantable medical device of this kind comprises a sensing device for sensing a magnetic field, and a processing device configured to detect the presence of an MRI device based on measurement values obtained from the sensing device.

An implantable medical device, for example, a stimulation device, such as a pacemaker device or a defibrillator device, generally is configured to output electrical stimulation energy for performing a therapeutic stimulation action. An implantable medical device in addition may be configured to sense electrical signals relating, e.g., to cardiac activity, in particular to trigger and clock a stimulation action. If an implantable medical device is introduced into a magnetic field of an MRI device, it can be assumed that electrical signals are induced on leads and other conducting parts of the implantable medical device, such electrical signals causing a disturbance of the operation of the medical device. For example, for an implantable medical device in the shape of a pacemaker device or a defibrillator device, a disturbance of a stimulation action may cause an erroneous stimulation and hence may have a significant impact on a therapy of a patient, which needs to be avoided.

There hence is a need to detect whether a patient carrying an implantable medical device is in the vicinity of an MRI device, such that, if this is the case, operation of the medical device may be modified in order to reduce a risk for a malfunction of the implantable medical device.

U.S. Pat. No. 9,364,663 B2 discloses an implantable medical device including a power supply, a sensing device and/or a stimulation device. An MR detection unit may identify an MR-typical journey of an implantable medical device on a patient bed of an MRI device, based on a change over time of measurement values of at least two magnetic field sensors and a difference between the measured values of the at least two magnetic field sensors. Herein, if measurement values of the at least two magnetic field sensors exceed a threshold, a presence of an MRI device is assumed.

U.S. Pat. No. 9,981,124 B2 discloses an implantable medical device having a first magnetic field direction sensor located at a first location within a housing and configured to generate a signal representative of a first direction of a magnetic field at the first location, and a second magnetic field direction sensor located at a second location within the housing and configured to generate a signal representative of a second direction of the magnetic field at the second location. Based on signals obtained via the first magnetic field direction sensor and the second magnetic field direction sensor it is concluded for a presence of an MRI device.

Generally, an implantable medical device is configured to interact electromagnetically with external devices, such as a programming device or a triggering device which are placed outside of a patient and can be brought into vicinity of an implantable medical device. By electromagnetic interaction of an external device with the implantable medical device a communication between the implantable medical device and the external device can be established, for example, for programming the implantable medical device or for transferring data from the implantable medical device to the external device.

An implantable medical device shall be configured to be able to distinguish between an external device, which shall interact with the implantable medical device, and another magnetic device, in particular an MRI device, which may cause a disturbance of an operation of the implantable medical device and hence needs to be detected in order to be able to suitably modify an operation of the implantable medical device in order to reduce a risk for a malfunction of the implantable medical device.

The present disclosure is directed toward overcoming one or more of the above-mentioned problems, though not necessarily limited to embodiments that do.

### SUMMARY

It is an object of the instant invention to provide an implantable medical device and a method for operating an implantable medical device which in an easy-to-implement way allow for a reliable detection of a presence of an MRI device.

At least this object is achieved by means of an implantable medical device comprising the features of claim 1.

Accordingly, the processing device of the implantable medical device is configured to conclude that an MRI device is present if a multiplicity of measurement values indicates an increase of a strength of the magnetic field.

An MRI device uses a strong, constant magnet field having, for example, a nominal magnetic field strength of above 1 T, for example, 1.5 T, 3 T or 7 T. By superimposing the constant magnetic field with time-varying magnetic gradient fields a magnetic resonant effect is induced, which may be detected using RF detection coils for picking up signals within a patient's body to conduct an imaging of the patient.

Herein, when a patient shall undergo an MRI examination, the patient typically is placed on a patient bed of the MRI device and, by moving the patient bed into a bore of the MRI device, is placed with a body part to be examined inside of the bore of the MRI device.

If a patient carrying an implantable medical device, such as a cardiac stimulation device, shall undergo an MRI examination, the implantable medical device shall be enabled to detect that the patient is approaching an MRI device such that operation of the implantable medical device may suitably be modified in order to reduce a risk of an impact of the MRI device, in particular the strong magnetic field of the MRI device, on the operation of the implantable medical device. If it is detected that a patient is in the range of the magnetic field of an MRI device, for example, a sensing function of the implantable medical device for triggering or clocking a stimulation action can be switched off, such that the implantable medical device, for example, may be operated in a mode using no sensing of electrical activity in a region of interest, for example, in a patient's heart, such that a therapy, for example, a pacing action, without a clocking based on external sensing signals is carried out (for example, in a so-called VV0 mode of a pacemaker device). In addition or alternatively, a therapy function may be switched off, for example, a shock function of a defibrillator device, such that an (erroneous) shock during an MRI examination is avoided.

For detecting whether a patient carrying an implantable medical device is in the vicinity of an MRI device, measurement values of a sensing device for detecting a magnetic field are examined. Herein, if it is found that a multiplicity of measurement values obtained from the sensing device of the implantable medical device indicate an increase of the strength of the magnetic field, it is assumed that a patient is placed on a patient bed of an MRI device and is moved, by moving the patient's bed with a substantially constant velocity, into the bore of the MRI device.

If a patient is placed on a patient bed of an MRI device and the patient bed is moved into the bore of the MRI device, the magnetic field strength at the location of the implantable medical device will steadily increase as the patient is moved into the MRI device. This steady increase of the magnetic field strength at the location of the implantable medical device may be observed by monitoring measurement values of the sensing device. If multiple temporally offset measurement values indicate an increase of the magnetic field strength, it may be concluded that the patient is moved into the MRI device, such that operation of the implantable medical device may suitably be modified.

Hence, by observing measurement values obtained from the sensing device and by determining whether an increase of the magnetic field strength indicative of a steady movement of the patient into an MRI device is present, a situation in which a patient is moved into an MRI device may be distinguished from a situation in which an external device other than an MRI device, such as a programming device or the like, is brought into the vicinity of the implantable medical device in order to interact with the implantable medical device. If a patient shall undergo an MRI examination, operation of the implantable medical device shall be monitored in order to avoid a malfunction. If instead an external device is brought into the vicinity of the implantable medical device, potentially a communication with the external medical device shall be established, or a particular function of the implantable medical device shall be triggered, the external device, for example, providing a wake-up signal to the implantable medical device.

In one embodiment, the sensing device is configured to provide measurement values at a predefined sampling rate. For example, using the sensing device measurements may be conducted at a specified frequency, for example, at a frequency in between 1 Hz and 50 Hz, for example, 4 Hz, such that the sensing device outputs (discrete) measurement values at the predefined sampling rate. The measurement values are input to the processing device, which analyzes the measurement values and, if multiple measurement values indicate a (steady) increase of the magnetic field strength it is concluded for the presence of an MRI device.

The sensing device may output measurement values indicative of the magnetic field strength. The measurement values herein may be output as digits, each digit corresponding to a specified voltage value, for example, 1 mV. As the sensing device may have a nonlinear characteristic curve, the voltage value may nonlinearly (dependent on the magnetic field strength) indicate a certain change in the magnetic field strength.

In one embodiment, the processing device is configured to conclude that an MRI device is present if a multiplicity of consecutive measurement values indicate an increase of the strength of the magnetic field. Hence, if it is found that for consecutive measurement values the magnetic field increases in strength, it is concluded that the patient is moved into an MRI device and hence an MRI device is identified.

For example, it may be concluded for the presence of an MRI device if for a multiplicity of consecutive measurement values an increase of the strength of the magnetic field exceeding, for each measurement value, a predefined margin is detected. Hence, if for a number of consecutive measurement values the magnetic field strength increases for each measurement value by at least a certain margin, it is concluded for the presence of an MRI device, as it is found that the strength of the magnetic field steadily increases due, for example, to a movement of a patient bed into the MRI device. Alternatively or in combination, the presence of an MRI device can be concluded if the magnetic field increases over a certain number of consecutive measurements without considerable drops.

In one embodiment, the processing device is configured to increment a counter value if a measurement value indicates an increase of the strength of the magnetic field. Herein, for example, the processing device may be configured to increment the counter value if a measurement value indicates an increase of the strength of the magnetic field exceeding a predefined count threshold. Hence, each time a measurement value obtained from the sensing device indicates that the strength of the magnetic field has changed by a certain minimum margin corresponding to the count threshold, the counter value is incremented, such that the counter value counts the times that the measurement values indicate a substantial increase of the magnetic field strength.

Herein, the counter value may be incremented by one from an initial start value, for example, 0, if the measurement value indicates an increase of the magnetic field strength exceeding a first margin. Subsequently, if subsequent measurement values indicate an increase of the magnetic field strength exceeding a second margin corresponding to the count threshold, for each increment the counter value is again increased by 1 such that the counter value indicates the number of measurement occasions at which the count threshold has been exceeded. The first margin may be larger than the second margin. If measurement values are output in digits, the first margin may, for example, correspond to a value between 2and 5 digits, for example, 3 digits, whereas the second margin corresponding to the count threshold may correspond to a value between 1 and 3 digits, for example, 2 digits.

The counter value hence is derived from the measurement values. The counter value in each case is incremented by one (if applicable) for a measurement value obtained from the sensing device, independent of the actual increase amount of the magnetic field strength.

According to an embodiment of the present invention, the processing device is configured to decrement the counter value if a measurement value indicates a decrease of the strength of the magnetic field, wherein the decrease of the magnetic field strength exceeds a predefined countdown threshold. For instance, if measurement values are output in digits, the countdown threshold may correspond to a drop of 2 or 3 digits.

According to an embodiment of the present invention, the counter value may not fall below zero.

The counter may be decremented in one step by one or more count values, e.g., two, three etc. By choosing a higher decrement count value, the counter is decremented faster. If the processing device is configured to perform certain actions in association with counter decrementation, e.g., withholding detection of the presence of an MRI machine or switching the implantable device from an MRI-mode back to normal operation, setting a higher decrement value (i.e., after a small number of counter decrements) will cause that actions to be triggered faster than if a smaller decrement value was chosen.

In one embodiment, the processing device is configured to conclude that an MRI device is present if the counter value is incremented for a predefined number of times (larger than 1, preferably larger than 2), for example, a predefined number of consecutive times. The predefined number of times, may, for example, have a value in between 2 and 20, for example, 5. If it is found that the counter value is steadily incremented for the predefined number of (consecutive) times, it is concluded for the presence of an MRI device, as it is found that the magnetic field strength at the location of the implantable medical device steadily increases.

In addition or alternatively, the processing device may be configured to conclude that an

MRI device is present if the counter value reaches or exceeds a predefined detection threshold. Hence, even if the counter value is not found to steadily increase over a predefined number of (consecutive) times, it may be concluded for the presence of an MRI device if the counter value reaches or exceeds the detection threshold. Hence, if the counter value becomes as large as or larger than the detection threshold, it in any case is assumed that an MRI device is present.

In one embodiment, the processing device is configured to reset the counter value to a start value, for example, 0, if the counter value is not incremented in a predefined period of time. The predefined period of time may, for example, range from 1 to 20 minutes, for example, 10 minutes. If it hence is found that the counter value no longer increases, which is observed over a suitable time span, the counter value is reset, because it is assumed that the implantable medical device is not or no longer in the range of an MRI device.

Alternatively or in addition, the processing device may be configured to reset the counter value to a start value, for example, 0, if a measurement value indicates a decrease of the strength of the magnetic field by a value larger than a predefined reset threshold. If it is found that the magnetic field strength substantially decreases, it can be assumed that the implantable medical device is not or no longer in the range of an MRI device. Hence, the counter value is reset such that a detection procedure is started anew to detect the presence of an MRI device in case of an increase of the magnetic field strength.

In an embodiment of the present invention, the processing device is configured to reset the counter value to a start value in case a predefined number of consecutive measurement values each indicate a decrease of the strength of the magnetic field. The counter value is reset if each of the measurement values show a decrease by a value which is larger than a predefined countdown threshold.

Setting predefined number of consecutive measurement values for deciding on counter reset has the advantage that an exit of an MRI environment of an MRI machine can be detected with a high reliability. By exiting the MRI environment, the magnetic field strength of the patient (and the implantable medical device) normally decreases in a controlled speed, which should be detected by the processing device. A single measurement of a decrease of the magnetic field followed by an increase, or single decreases alternating with increases, would in some cases not be sufficient to indicate an exit from the MRI environment. For instance, such events could be caused by a disturbance of the magnetic field measurements, or by an adjustment of the patient table of the MRI by the physician or nurse. For example, the number of consecutive measurement values can be set to 2 to 10 measurements. Particularly reliable results could be produced when the number was set to 3 consecutive measurements. The predefined countdown threshold may correspond to a drop of 2 or 3 digits.

In one embodiment, the sensing device may be a GMR sensor (GMR stands for giant magnetoresistance). The sensing device hence is configured to measure a magnetic field strength and output a voltage value indicative of a magnetic field strength present at the location of the sensing device.

In one embodiment, the processing device is configured to modify operation of the implantable medical device if it is concluded that an MRI device is present. The modification of operation may in particular include a switching-off of a sensing function of the implantable medical device, or a switching-off or modification of a therapy function. For example, for a stimulation device such as a pacemaker device a pacing function may be modified such that a pacing without an external trigger or clocking is achieved, for example, corresponding to a so-called VV0 mode of a pacemaker device. For a defibrillator device, for example, a shock function may be switched off such that a shock function is not available while a patient carrying the device undergoes an MRI examination.

According to an embodiment of the present invention, the processing device is configured to terminate said modifying of operation of the implantable medical device if the counter value is reset. A reset of the counter value is associated with exit of the MRI environment, so that modification of the operation of the implantable device becomes unnecessary, the implantable device may return to a normal non-MRI mode.

According to an embodiment, the processing device is configured to start a termination timer if counter value is reset. The modifying of operation of the implantable medical device is terminated as the termination timer reaches a predefined time margin. Applying a termination timer is advantageous for situations in which the counter value is reset before the patient exits the MRI environment entirely. In such cases, the modification of the operation of the implantable medical device is maintained after counter value reset for a certain time span as a safety measure for the patient. The time span is defined by said time margin and can be set to, e.g., 30, 60, 90, 120 seconds. In particular, setting the time margin to 60 seconds produced reliable results. If during that time span the counter value increases again, so that presence of an MRI device is redetected, the modifying of the operation of the implantable device may be maintained (i.e., not terminated after expiry of the time span). If no presence of an MRI device is redetected during said time span or until the termination timer reached the time margin, the modifying of the operation of the implantable device is terminated and the implantable medical device returns to a normal, non-MRI operational mode. Back to the normal operational mode, the implantable medical device resumes to apply the routines for detecting the presence of an MRI machine according to the present invention. The termination timer is set to its start value and will be retriggered next time the counter value is reset.

Alternatively, according to an embodiment of the present invention, the processing device is configured to conclude that an MRI device is present if at least a part of a curve associated with the multiplicity of consecutive measurement values corresponds to at least a part of a reference curve. In the context of the present invention, a part of a curve corresponds to a part of a second curve if their shapes are similar by a certain degree. For example, a curve generated by the multiplicity of consecutive measurement values obtained by the sensing device can be compared to a reference curve by calculating the difference of gradients of a part of both curves, and comparing the difference to a threshold. In case the difference stays below the threshold, the two parts of the curves may correspond to each other. If the difference exceeds the threshold, there may be no correspondence between the sections of both curves. Alternatively, the parts of both curves can be compared by calculating the difference between the areas under the curves. If the difference stays below a certain threshold, the two parts of the curves may have a certain degree of similarity and they correspond to each other. The reference curve may be stored in the implantable medical device in the manufacturing process, and is accessible to the processing device. For instance, the reference curve may be generated via numerous measurements of the magnetic field strength of an object which enters an MRI machine on a patient table, generating numerous test curves. The test curves are used to compute an average curve, which is stored as reference curve. As an alternative or in combination, the reference curve may be adapted or updated continuously by newly generated curves associated with the multiplicity of consecutive measurement values from the sensing device.

At least the object is also achieved by a method for operating an implantable medical device, the method comprising: sensing a magnetic field using a sensing device of the implantable medical device, and detecting a presence of an MRI device, using a processing device of the implantable medical device, based on measurement values obtained from the sensing device. Herein, it is concluded, by the processing device, that an MRI device is present if a multiplicity of measurement values indicates an increase of a strength of the magnetic field.

At least the advantages and advantageous embodiments of the implantable medical device as described above equally apply also to the method, such that it shall be referred to the above.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

Various features and advantages of the present invention may be more readily understood with reference to the following detailed description and the embodiments shown in the drawings. Herein,

FIG. 1 shows a schematic illustration of an implantable medical device in a patient;

FIG. 2 shows a schematic drawing of an MRI device;

FIG. 3 shows a schematic drawing of an implantable medical device;

FIG. 4 shows graphs of measurement values output by a sensing device of the implantable medical device, and a counter value derived from the measurement values; and

FIG. 5 shows characteristic curves of a sensing device in the shape of a GMR sensor.

### DETAILED DESCRIPTION

Subsequently, embodiments of the present invention shall be described in detail with reference to the drawings. In the drawings, like reference numerals shall designate functionally similar structural elements, if appropriate.

It is to be noted that the embodiments are not limiting for the present invention, but merely represent illustrative examples.

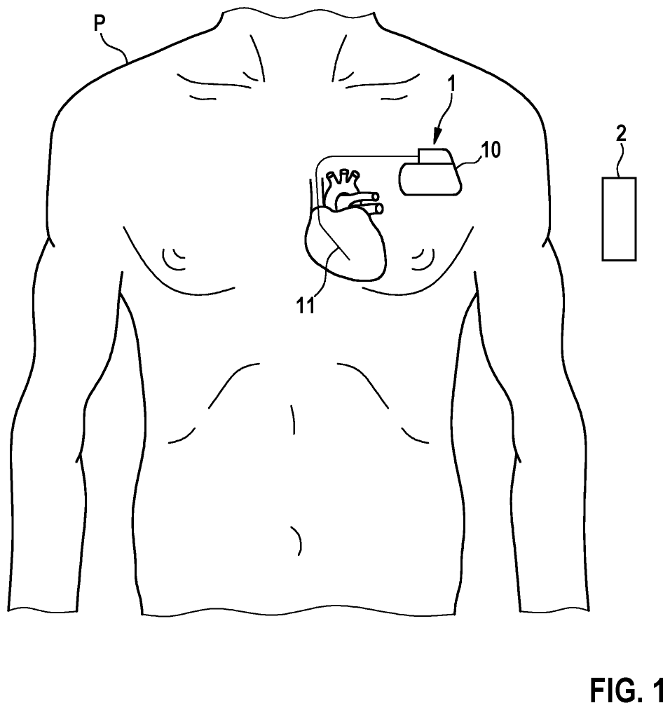

FIG. 1 shows a schematic illustration of an implantable medical device 1, for example, in the shape of a stimulation device, such as a pacing device or a defibrillation device. The implantable medical device 1 may, for example, comprise a generator 10 which, for example, as illustrated in FIG. 1, may be subcutaneously implanted into a patient, wherein an electrode 11 is connected to the generator 10 and extends from the generator 10 towards a region of interest, for example, the patient's heart, such that a therapy function may be provided at the region of interest, for example, in the patient's heart.

An implantable medical device 1 of this kind may, for example, be configured to provide a therapy function over a prolonged period of time, for example, a pacing function or a defibrillator function. The implantable medical device 1, for this, may be permanently implanted into a patient P and may function in a substantially autarkic manner, wherein a communication connection may be established with the implantable medical device 1 using an external device 2, for example, to program the implantable medical device 1 or to transfer, using, for example, telemetry, data from the implantable medical device 1 to the external device 2.

An external device 2 may communicate with the implantable medical device 1 using electromagnetic means, for example, by establishing an inductive coupling in between the implantable medical device 1 and the external device 2.

The external device 2 may alternatively be a permanent magnet which may be brought into the vicinity of the implantable medical device 1 in order to trigger an action of the implantable medical device 1, for example, for waking up the medical device 1.

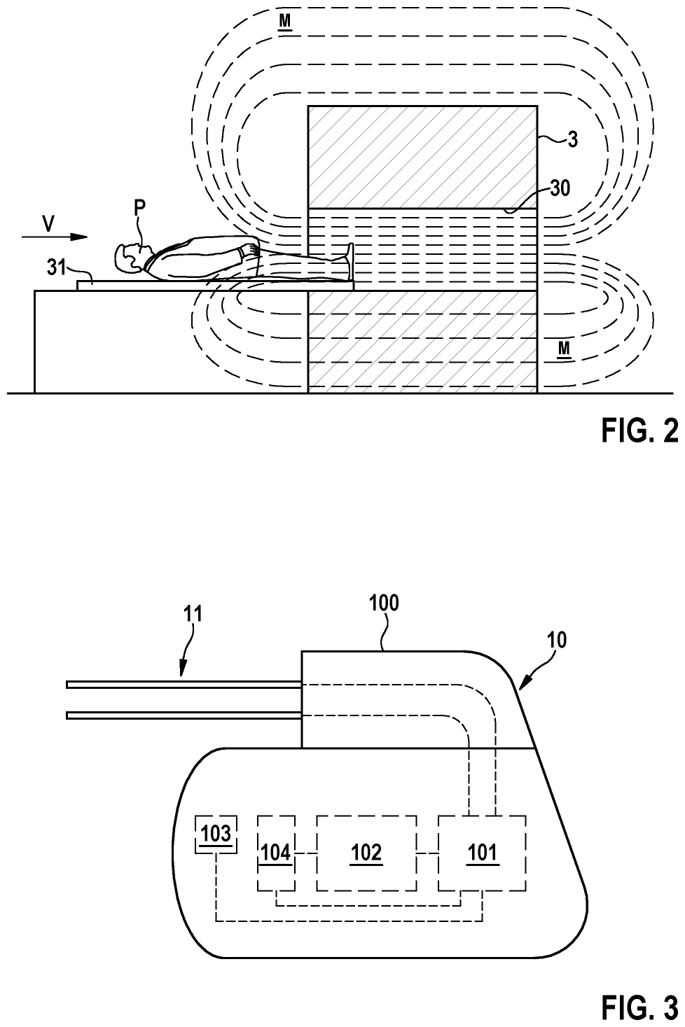

If, as schematically illustrated in FIG. 2, a patient P shall undergo an MRI examination using an MRI device 3, the patient P, carrying an implantable medical device 1, is introduced into a bore 30 of the MRI device 3 by placing the patient P on a patient bed 31 and by continuously moving, using an electro-motoric drive of the patient bed 31, the patient P into the bore 30 in a movement direction V. When moving the patient P into the bore 30 of the MRI device 3, the patient P herein is brought into the range of a constant magnetic field M of the MRI device 3, the magnetic field M generally having a maximum magnetic field strength (corresponding to the magnetic flux density B and indicated usually in Tesla [T]) within the bore 30. Hence, when the patient P is moved into the bore 30 of the MRI device 3, the magnetic field strength of the magnetic field M at the location of the implantable medical device 1 will steadily increase.

Caused by a magnetic field M of an MRI device 3, electrical signals may be induced within an implantable medical device 1. Hence, it shall be detected if an implantable medical device 1 comes into the range of an MRI device 3, such that operation of the implantable medical device 1 can suitably be modified in order to avoid a disturbance of operation by the MRI device 3.

FIG. 3 illustrates an embodiment of a generator 10 of an implantable medical device 1, for example, in the shape of a stimulation device such as a pacemaker device or a defibrillator device. Included in a housing of the generator 10 is a processing device 101, implemented, e.g., by electronic circuitry on a circuit board, which serves to control operation of the generator 10 for transmission of electrical stimulation energy via the electrodes 11 connected to a connector block 100 of the generator 10, and for analyzing sense signals received, e.g., via the electrodes 11 to provide for a therapy aligned to an activity of, for example, the patient's heart.

The generator 10 further comprises an energy storage 102 in the shape of a battery, a sensing device 103, for example, in the shape of a GMR sensor for sensing magnetic fields, and a communication device 104 for establishing a communication connection, for example, to an external device 2, as illustrated in FIG. 1.

The sensing device 103 is connected to the processing device 101 and is configured to conduct measurements yielding measurement values indicative of a magnetic field strength at the location of the sensing device 103. The sensing device 103 may, for example, be configured to conduct measurements at a specified sampling rate, for example, at a rate in between 1 Hz and 50 Hz, for example, 4 Hz. The sensing device 103 provides (discrete) measurement values to the processing device 101, which are analyzed by the processing device 101 and are used for controlling operation of the implantable medical device 1.

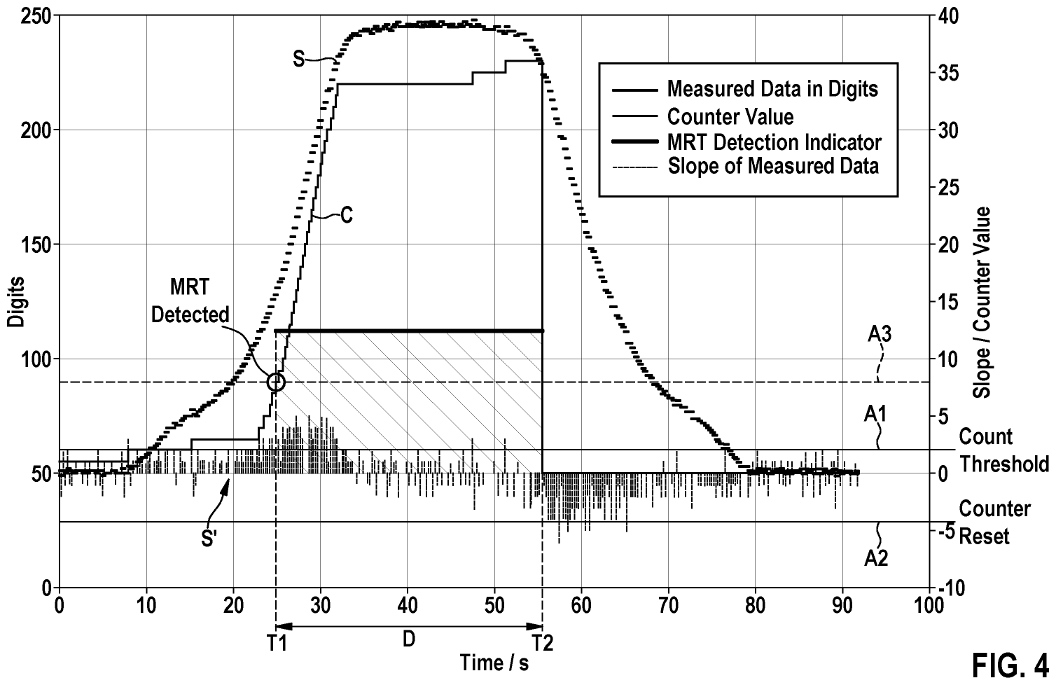

Referring now to FIG. 4, when a patient P is moved into the bore 30 of an MRI device 3 by electromotively moving the patient's bed 31 in a movement direction V, the sensing device 103 outputs measurement values S over time. The measurement values S herein are provided at the specified sampling rate in digits, one digit corresponding to a voltage value of 1 mV as output by the sensing device 103 (left vertical axis in FIG. 4).

FIG. 4 in addition to the measurement values S shows corresponding change values S′ (corresponding to the discrete slope of the measurement values S) over time, the change values S′ indicating a change between an actual measurement value and the previous measurement value (right vertical axis in FIG. 4).

Based on the measurement values S and the change values S′ a counter value C is derived.

Herein, the counter value C initially is set to 0. If a measurement value S is obtained which corresponds to a change value S′ exceeding a specified initial margin, for example, 3 digits, the counter value is incremented from 0 to 1, wherein subsequently the counter value C is incremented by one each time the change value S′ associated with the actual measurement value S exceeds a predefined count threshold A1, corresponding, for example, to a value of 2 digits.

If it is found that a counter value C reaches, at time T1, a detection threshold A3, it is assumed that an MRI device 3 is present and hence a flag “MRI detected” may be set by the processing device 101. Accordingly, in a phase D following time T1 operation of the implantable medical device 1 may be modified in order to account for the presence of the magnetic field M of the MRI device 3 and to suitably adjust operation of the implantable medical device 1 in order to reduce a risk for disturbances caused by the magnetic field M of the MRI device 3.

Alternatively or in addition to comparing the counter value C to a detection threshold A3, the processing device 101 may be configured to determine whether the counter value C for a predefined number of consecutive times is incremented, indicating that the change value S′ for consecutive measurement values S repeatedly exceeds the count threshold A1. The predefined number of consecutive times may, for example, be set to 5. If, hence, the counter value C is consecutively incremented by 1 for five consecutive measurements, it is assumed that presumably the magnetic field steadily increases as the patient P is moved into an MRI device 3 and, hence, an MRI device 3 is present.

In the example of FIG. 4, at time T1 an MRI device 3 is assumed to be detected, and hence in phase D following time T1 a suitable indicator flag is set. The counter value C, due to the further increase of the magnetic field M and the corresponding rise of the measurement values S, continues to be incremented, until a plateau is reached, corresponding, for example, to a rest period in which the patient P rests in the bore 30 of the MRI device 3 and the patient bed 31 is at a standstill.

If the patient P is moved out of the bore 30, the magnetic field strength at the location of the implantable medical device 1 will decrease, which is detected and indicated by the measurement values S. If it is found that a change value S′ corresponding to a measurement value S indicates a decrease of the magnetic field strength by a margin exceeding a reset threshold A2, as it is the case at time T2 in FIG. 4, the counter value C is reset to 0. In addition, the “MRI detected” indicator flag is canceled, such that the phase D indicating a detected MRI device 3 lasts between time T1 and time T2, as illustrated in FIG. 4.

Alternatively or in addition, if it is found that the counter value C is not further incremented over a predefined period of time, for example, ranging from 1 minute to 20 minutes, the counter value C may also be reset to 0.

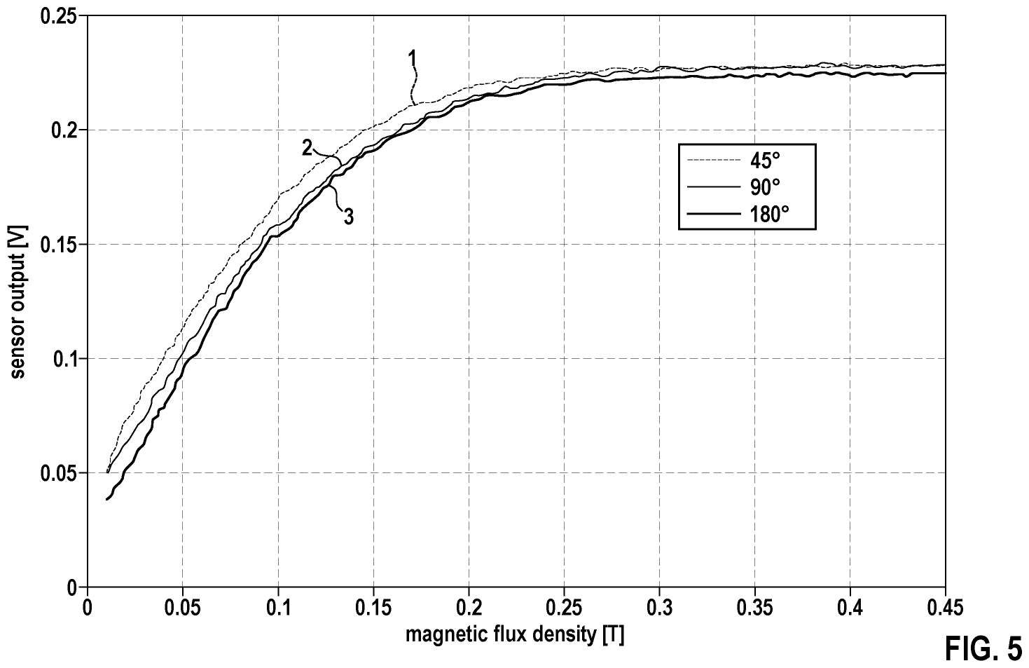

Referring now to FIG. 5, the sensing device 103, for example, implemented using two GMR sensors rotated by 90° to one another, has characteristic curves O1, O2, O3 which are nonlinear and are substantially independent of an angular orientation of the sensing device 103 (illustrated by the fact that the characteristic curves O1, O2, O3 corresponding to different angular orientations of the sensing device 103 are substantially identical). The sensing device 103 outputs digits, each digit corresponding to a voltage value of 1 mV. Herein, the digits are nonlinearly correlated to the magnetic field strength (magnetic flux density, measured in Tesla), as this is indicated by the characteristic curves O1, O2, O3.

If the sensing device 103 detects the presence of an MRI device 3 and correspondingly sets the “MRI detected” flag, the processing device 101 is configured to modify operation of the implantable medical device 1. For example, if the implantable medical device 1 is a stimulation device, for example, a pacemaker device, a therapy function can be modified, for example, by switching off a sensing function in order to provide a pacing action without a clocking by cardiac activity. If the implantable medical device 1 is, for example, a defibrillator device, a shock function may be switched off, in particular in order to avoid an erroneous shock while a patient P is within an MRI device 3, due to signals induced within the implantable medical device 1 by the magnetic field M of the MRI device 3.

An implantable medical device as concerned herein may in particular be a cardiac stimulation device such as a pacemaker device or a defibrillator device, but may also be a stimulation device, for example, for a neuro-stimulation. In addition, the implantable medical device may, for example, be a sensor device, such as an implantable pressure sensor or the like.

Because a magnetic field strength, for determining whether an MRI device is present, is analyzed over time by observing multiple (preferably consecutive) measurement values, a detection reliability for detecting an MRI device—in differentiation to another external device—may be improved. In this way the number of wrong detections of an MRI device may be substantially reduced, hence improving therapy for a patient and the patient's well-being.

A functionality as described herein may be implemented by a change in software of an implantable medical device, such that implementation may be easy and cost-effective without requiring a change in hardware.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Implantable medical device (pacemaker device)
10 Generator
100 Connector block
101 Processing device
102 Energy storage
103 Sensing device
104 Communication device
11 Electrode
2 External device
3 MRI device
30 Bore
31 Patient bed
A1 Count threshold
A2 Reset threshold
A3 Detection threshold
C Counter value
D Phase
M Magnetic field (magnetic flux density)
O1, O2, O3 Characteristic curve (sensor output)
P Patient
S Measurement values (in digits)
S′ Slope
V Moving direction

1. An implantable medical device, comprising: a sensing device for sensing a magnetic field; and a processing device configured to detect a presence of an MRI device based on measurement values obtained from the sensing device; wherein the processing device is configured to conclude that an MRI device is present if a multiplicity of measurement values obtained from the sensing device indicates an increase of a strength of the magnetic field, wherein the sensing device is configured to conduct measurements at a specified frequency in between 1 Hz and 50 Hz, and to provide measurement values at a predefined sampling rate.

2. The implantable medical device of claim 1, wherein the processing device is configured to conclude that an MRI device is present if a multiplicity of consecutive measurement values indicate an increase of the strength of the magnetic field.

3. The implantable medical device of claim 1, wherein the processing device is configured to increment a counter value if a measurement value indicates an increase of the strength of the magnetic field.

4. The implantable medical device of claim 3, wherein the processing device is configured to increment the counter value if a measurement value indicates an increase of the strength of the magnetic field exceeding a predefined count threshold.

5. The implantable medical device of claim 3, wherein the processing device is configured to decrement the counter value if a measurement value indicates a decrease of the strength of the magnetic field, wherein the decrease of the magnetic field strength exceeds a predefined countdown threshold.

6. The implantable medical device of claim 3, wherein the processing device is configured to conclude that an MRI device is present if the counter value is increased for a predefined number of times or if the counter value reaches or exceeds a predefined detection threshold.

7. The implantable medical device of claim 6, wherein the processing device is configured to conclude that an MRI device is present if the counter value is increased for a predefined number of consecutive times or if the counter value reaches or exceeds a predefined detection threshold.

8. The implantable medical device of claim 3, wherein the processing device is configured to reset the counter value to a start value if the counter value is not increased in a predefined period of time.

9. The implantable medical device of claim 3, wherein the processing device is configured to reset the counter value to a start value if a measurement value indicates a decrease of the strength of the magnetic field by a value larger than a predefined reset threshold.

10. The implantable medical device of claim 3, wherein the processing device is configured to reset the counter value to a start value if a predefined number of consecutive measurement values each indicate a decrease of the strength of the magnetic field by a value larger than a predefined countdown threshold.

11. The implantable medical device of claim 1, wherein the processing device is configured to modify operation of the implantable medical device if it is concluded that an MRI device is present.

12. The implantable medical device of claim 5, wherein the processing device is configured to:
modify operation of the implantable medical device if it is concluded than an MRI device is present, and
terminate modification of operation of the implantable medical device if counter value is reset, or
start a termination timer if counter value is reset, and terminate modification of operation of the implantable medical device when the termination timer reached a predefined time margin.

13. The implantable medical device of claim 1, wherein the processing device is configured to conclude that an MRI device is present if a at least a part of a curve associated with the multiplicity of consecutive measurement values corresponds to at least a part of a reference curve.

14. Method for operating an implantable medical device, comprising:
sensing a magnetic field using a sensing device of the implantable medical device; and detecting a presence of an MRI device, using a processing device of the implantable medical device, based on measurement values obtained from the sensing device; concluding, by the processing device, that an MRI device is present if a multiplicity of measurement values obtained from the sensing device indicates an increase of a strength of the magnetic field, wherein the sensing device conducts measurements at a specified frequency in between 1 Hz and 50 Hz, and provides measurement values at a predefined sampling rate.

15. The implantable medical device of claim 1, wherein the specified frequency is 4 Hz.

16. The method of claim 14, wherein the specified frequency is 4 Hz.
